(19) United States
(12) Patent Application Publication
(10) Pub. No.: US 2017/0156653 A1
(43) Pub. Date: Jun. 8, 2017

(54) Blood Collection Apparatus and Method

(71) Applicant: Taneesha Sharee Scott, West Berlin, NJ (US)

(72) Inventor: Taneesha Sharee Scott, West Berlin, NJ (US)

(21) Appl. No.: 14/956,462

(22) Filed: Dec. 2, 2015

(51) Int. Cl. A61B 5/155; A61B 5/15

(52) U.S. Cl. CPC A61B 5/155; A61B 5/15074; A61B 5/150343; A61B 5/150473; A61B 5/150732; A61B 5/150534

(57) ABSTRACT

An apparatus and method of collecting multiple vials of blood specimens simultaneously, wherein all vials are filled from a single patient insertion site so that upon completion from one to three vials of blood have been collected.

### FIELD OF THE INVENTION

The present invention relates to an apparatus and a process for using the same for the collection of blood specimens. The invention further relates to an apparatus and process for the collection of multiple samples of blood. Even further, the present invention is an improved blood collection system that is designed to use the hemodynamic process of the body to facilitate blood collection. More specifically, the present invention provides both an apparatus and method for utilizing the same wherein, prior to acting as a blood collection system apparatus must be assembled and snapped into place before one to multiple blood samples are provided from a single needle insertion site.

### BACKGROUND OF THE INVENTION

Improved blood collection systems are being required to meet the growing demand for blood collection methods that make it possible to collect multiple venous samples simultaneously during the venipuncture process. The method and apparatus of the present invention may be utilized wherever the need to collect venous samples, and because of its portability, is capable of use in places, conditions and circumstances were traditional blood collection systems are inadequate due to size, shape and/or method of utilization.

In accordance with the above listed objects, other objects, such as the present apparatus portability, will become more apparent in the course of this invention disclosure as is further and more substantially describe herein and finally claimed hereinafter.

### SUMMARY OF THE INVENTION

The current invention relates to an apparatus and a process for using the apparatus to simultaneously collect multiple blood samples from a patient from a single needle insertion site. More particularly it relates to an apparatus that has multiple parts including a butterfly needle system, a Y-configured splitter bridge and a vial holder.

In the current process, the splitter collects blood samples in evacuated vials/tubes and the holder is configured for securing the evacuated vials/tubes while the blood is being collected therein. The method allows for the collection of a single blood sample or multiple blood samples, meaning from a single sample to a total of three (3) blood samples from a single insertion site. However, for proper operation, the Y-configured splitter bridge and holder must be properly aligned and securely attached prior to the collection of blood from the patient.

The current apparatus is capable of collecting from one to three vials of blood simultaneously, while utilizing a single insertion point. During the collection process, a Y-shaped or Y-configured splitter bridge divides and separates the blood flow into three separate and equal blood streams, filling the vials simultaneously. The apparatus creates an evenly divided hemodynamic process to mimic the same process internal to the body where, for example, a main artery divides and branches out into many smaller arteries so that each vial housed and secured by the holder, has its own source and flow of blood.

The apparatus facilitates the simultaneous flow of blood from one to three evacuated tubes or vials as opposed to a succession blood collection system. By simultaneous the evacuated vials are filled from a single patient insertion site, at the same time, at the same blood flow rate, so that the vials fill evenly. Succession blood means that one tube or vial in a multiple series of evacuated tubes must be filled prior to any blood flow to the next, adjacent or any subsequent tube in the same multiple series of evacuated tubes. The term tube and vial are used interchangeably throughout the application.

### BRIEF DESCRIPTION OF THE DRAWINGS

FIG. 1, shows a version of the butterfly needle system and splitter bridge assembly.

FIG. 2, shows a version of the Y-configured splitter bridge.

FIG. 3, shows a version of the attaching means with the evacuated vial holder.

FIG. 4, shows a version of the evacuated vials and evacuated vial holder.

### DETAILED DESCRIPTION

The current invention relates to an apparatus and a process for the simultaneous collection of multiple blood samples from a single insertion site on a patient.

In one aspect, the current apparatus includes a butterfly needle system, wherein the butterfly needle system includes a dual open-end needle, a base for the needle and two butterfly wings. The apparatus further consists of memory free tubing, a female luer lock connector, a male luer lock connector, a Y-configured splitter bridge, leg extensions, extended pieces of plastic, rubber sleeve caps, a second dual open-end needle, rubber sleeves, and an attaching means. The apparatus further consists of a holder that attaches to the attaching means of the Y-configured splitter bridge and is capable of securing from one to three evacuated vials.

In one aspect of the above apparatus, there is a Y-configured splitter bridge and system holder, such that, after attaching the butterfly needle system to the Y-configured (this shouldn't be here) male luer lock adaptor on one end and the vials holder to the other end of the Y-configured splitter bridge, multiple vials of blood can be simultaneously collected from a single insertion site.

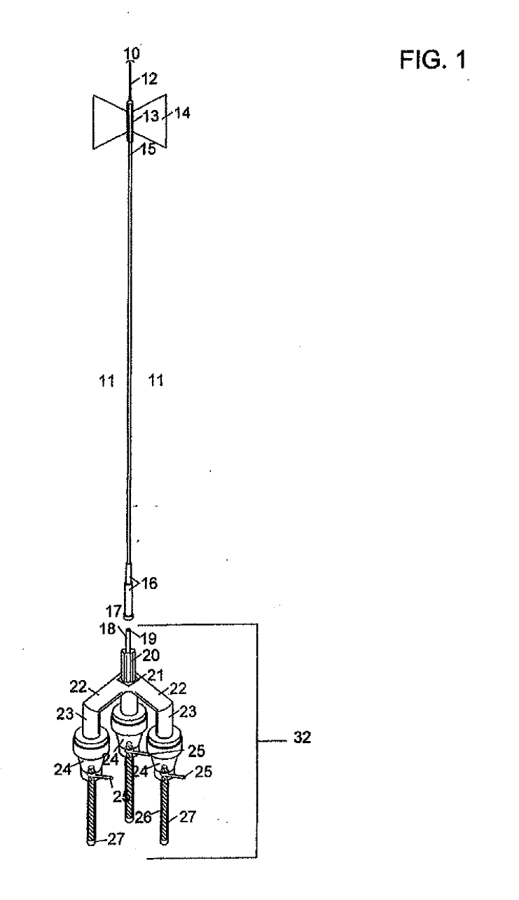

In one aspect of the current device, FIG. 1 shows the apparatus including a butterfly needle system (11) and splitter bridge assembly (32). The apparatus is capable of collecting one, two, or three samples simultaneously from a single insertion site (10). The butterfly needle system (11) includes a first dual open-end needle (12) that can be 21-23 gauge, a base for the needle (13) and butterfly or flexible wings (14). In addition, it includes memory free tubing wherein one end of memory free tubing (15) can be connected to the dual open-ended needle (12) at one end, and the opposite end can be connected to a female luer lock connector (16). The female luer lock connector (16) has a hollow middle core defining an open-end bottom, which can be attached to a male luer lock connector system (17), which is mounted to a Y-configured splitter bridge (21). The dual open-end needle (12) can be cylindrical in shape with dual open-ends and can having a lumen size of 21-23 gauge and wherein needle (12) length is from about ½ in (1.27 cm) to about ¾ in (1.91 cm) in length.

In FIG. 1, the Y-configured splitter bridge (21) a male luer lock connector unit (17) that is mounted or otherwise attached to the Y-configured splitter bridge and can therefore be thought of as a part of the male luer lock connector unit (17) includes a predisposed extension tip (18), an open core capillary (19), and a male luer lock base (20) that is mounted on the Y-configured splitter bridge (21) and allows for the flow of blood through the Y-configured splitter bridge. The Y-configured splitter bridge (21) can be made from any malleable material such as metal, polypropylene (plastic) and the like. Y-configured splitter bridge (21), can further includes predisposed leg extensions (22) and extended leg pieces (23), rubber sleeve caps (24), connector means (25), a second dual open-end open needle (26), and a rubber sleeve (27). The Y-configured splitter bridge (21) is supplied blood from the patient through the butterfly needle system (11) at a constant flow through the mounted male luer lock connector unit (17) which is aligned with the opposite end of the memory free tubing (15) within and inside of the female luer lock connector (16). The blood comes from a single insertion site (10) through the memory free tubing (15) to the female luer lock connector (16) and male luer lock connector unit (17) mounted on top of the Y-configured splitter bridge (21). From here, the predisposed extension tip (18) is able to accept blood flow through the female luer lock connector (16).

In FIG. 1, rubber sleeve cap (24) is configured with an attaching or connector means (25), such as a snap, a second dual open-end needle (26), and a rubber sleeve (27) for covering the second dual open-ended needle (26). The second dual open-end needle (26) is inserted within and inside middle of each rubber sleeve cap (27). The second dual open-end needle (26) is further utilized to complete two functions; the first function is to accept blood at one end, after the blood passes through the rubber sleeve cap (24) which are mounted to the bottom end of the extended leg pieces (23) which are configured downward at approximately 90 degree angle. The second function of the second dual open-end needle (26), is to pierce a puncture stopper (37) mounted on the evacuated vials (39) which can have an approximate diameter of about 13 mm to about 16 mm (250/sp, 1000/ca), a length of about 13 inches (33 cm) to about 17.7 inches (45 cm) and width of 9.0 inches (23 cm) to 13.78 inches (43 cm) in order to finally deliver and discharge blood flow into the evacuated tube(s) or vial(s) (39), being held by evacuated vial holder (34).

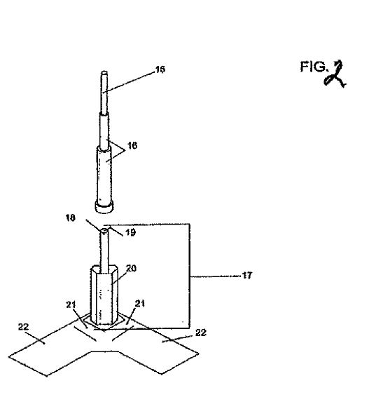

In yet another aspect, the device as shown in FIG. 2 consists of memory free tubing (15) with a capable volume of about 0.25 milliliter (mL) to about 0.45 mL, a female luer lock connector (16), a male luer lock connector unit (17) that consists of a predisposed extension tip (18), having, an open core capillary (19), a male luer lock base (20), Y-configured splitter bridge (21) and leg extensions (22) which are longer in length than they Y-configured splitter bridge (21). The Y-configured splitter bridge (21) consists of a male luer lock connector (17). The splitter (32) is made from any malleable material such as polypropylene (plastic) and the like. The male luer lock connected unit (17) is permanently mounted on the Y-configured splitter bridge (21) and not detachable from the splitter bridge unit.

In some aspects, the Y-configured splitter bridge (21) is supplied blood at a constant flow through the mounted male luer lock connector unit (17), and the blood simultaneously supplied to each individual evacuated vial (39).

In other aspects, the memory free tubing (15) has a length from about 30.5 mm (about 12 inches) and one end is inserted into the base (13) while the other end is connected with the female luer lock connector (16). From a single insertion site (10) of a patient, blood is fed through the butterfly needle system (11), through the splitter bridge assembly and finally into the evacuated vials (39).

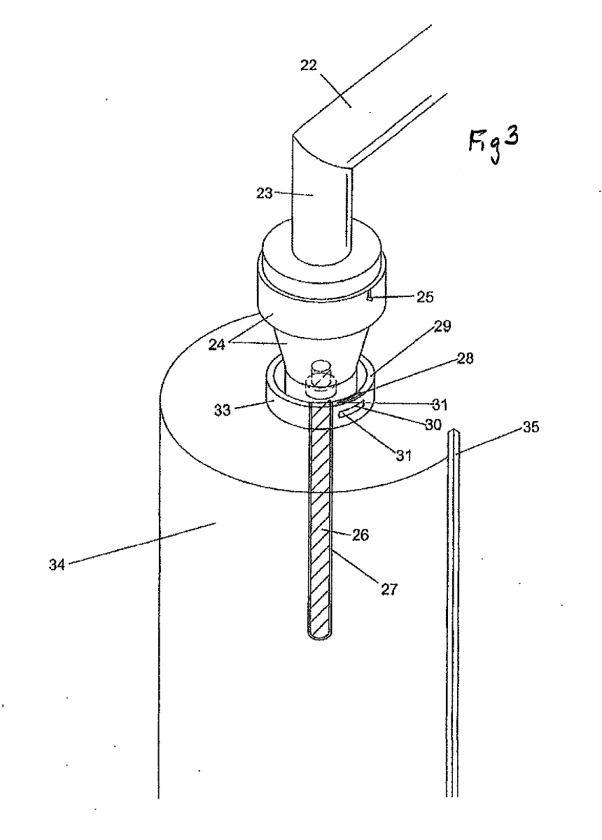

In yet other aspects of the device, FIG. 3 illustrates the Y-configured splitter bridge is further configured with leg extensions (22), extended leg pieces (23), a rubber sleeve cap (24), an attaching means or snap (25), a second dual open-end needle (26) and a rubber sleeve (27). Wherein, the rubber sleeve cap (24) is configured to accept a second dual open-end needle (26) is also further configured with a connecting means, such as, a snap (25) for attaching the splitter bridge system (32) to an evacuated vial holder (34). The rubber sleeve cap (24) can be made out of non-impervious material such as plastic.

In other aspects of the device, the splitter bridge system (32) is configured to accept a rubber sleeve (27) which is used to substantially cover and seal the second dual open-end needle (26). The second dual open-end needle (26) is mounted to the center of rubber sleeve cap (24), wherein rubber sleeve cap (24) is also further configured with a connecting means (25), such as a snap, for connecting or attaching the splitter bridge system (32) to the evacuated vial holder (34). The opposite end of the second dual open-end needle (26) lies in wait for evacuated vials (39) to be inserted into the evacuated vial holder (34).

In yet other aspects of the device, FIG. 3 shows the second dual open-end needle (26) and rubber sleeve (27) and cap (24) attached to the extended pieces (23) having a connecting means, such as a snap (25). One end of the second dual open-end needle is attached to the rubber sleeve cap (24) as described above. There is a exterior base cut out (28) is located at the rim (29) of evacuated vial holder (34), while an interior locking station (30) is positioned directly below the exterior base cut out (28) between the vertical and horizontal walls of neck (33) of the evacuated vial holder (34). There are two easy guide slits (31) that run parallel, extending from the left of the exterior base cut out (28) at the rim (29) and the left of the interior locking station (30) between vertical and horizontal walls of neck (33) of the evacuated vial holder (34).

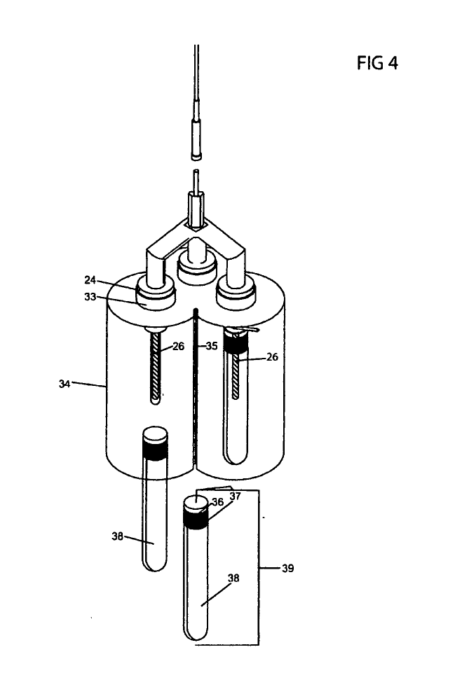

In some aspects of the device an evacuated vial set-up (39) as shown in FIG. 4, is provided wherein vials (38) are evacuated causing a negative pressure inside the vial. The evacuated vial set-up (39) includes a vial (38), an evacuated top (36) and a puncture stopper (37). The evacuated vial holder (34) can have the shape of a three leaf clover and is connected or attached to the splitter bridge assembly (32). second

In yet other aspects of the device, the rubber sleeve (27) is designed to maintain sterilization of the second dual open-end needle (26) and to collapse the instant the puncture stopper (37) is pushed onto the second dual open-end needle (26).

In some aspects of the current method, the puncture stopper (37) is pushed against and pierced by the second dual open-end needle (26). The evacuated vials (39) can have a height of about 6.8 inches (17 cm) to about 12.2 inches (30 cm) and a weight of about 0.143 ounces (oz) (4 gms) to about 8.29 pounds (235 gms) for the purpose of delivering blood into one to three evacuated vials. The air pressure inside the evacuated vial set-up (39) is negative, less than the normal or atmospheric pressure. The difference between pressure inside of the evacuated vial set-up (39) and the vein causes blood to fill the vial (38). The vials contain air pressure that is negative than normal air and therefore fill simultaneously and automatically once blood collection is started, wherein a dual open-end needle (12) is used on a patient at a single insertion site (10), with the bevel of the dual open-ended needle (12) positioned upward and angled at approximately 45 degrees, thereby creating a system for collecting one to three samples of blood.

In some aspects of the current method, a licensed clinician will position the patient with his/her arm extended. The patient's wrist should be lower than the bend in the arm. A tourniquet can be applied at about three inches above the selected single insertion site (10). Sterilize the single insertion site (10) with alcohol wiping in a circular motion. While positioning a dual open-ended needle (12) with bevel-side facing up at the time of insertion, swiftly insert needle (12) through the skin at an approximate forty-five degree angle. The butterfly needle system (11) makes vein-puncture easier with automatic alignment of the needle (12) in bevel-up position.

In some aspects of the current device, the base (13) of the butterfly needle system (11) further consists of two flexible wings (14), so that, when the flexible wings (14) are held together by the technician and utilized as a grip, a steady insertion of needle (12) into the patient's vein when drawing blood is easily accomplished.

In some aspects of the device, the female luer lock connector further has a hollow middle core defining an open-end bottom, which is attached firmly to the male luer lock connector unit (17) which is mounted to the Y-configured splitter bridge (21).

In some aspects of the current device, the female luer lock connector (16) is about 30.5 millimeters (mm) (about 12 inches) in length, and the tubing volume can be from about 0.25 ml to about 0.45 ml. The flexible memory tubing (15) can be a translucent, latex-free, super-flex tubing, which allows a patient some degree of mobility during blood collection without upsetting the butterfly needle system (11) and flexible memory tubing (15) attachment,

In yet other aspects of the device, the leg extensions (22) are mounted to the Y-bridge splitter (21) and the leg extensions (22) have additional extended leg pieces (23). Leg extensions (22) are hollow and are an extension of the Y-configured splitter bridge (21), wherein the blood flows from the patient to the Y-configured splitter bridge (21) and the leg extensions (22) receives the blood from the Y-configured splitter bridge (21). The blood flows through the leg extensions (22), which can be configured with approximately 45 degree bends in order to accommodate blood flow with gravity from the splitter bridge (21), and through the extended leg pieces (23), which are also hollow, that are connected to the leg extensions at approximate a 90 degree angle, thereby keeping the flow of blood moving in a continuous, smooth and uninterrupted flow in order to simultaneously fill the evacuated vials (39).

In some aspects, the rubber sleeve cap (24) is a rigid malleable material, such as polypropylene, configured to accept and mount a 21-23 gauge needle that is a second dual open-end needle (26), which can accept the blood flowing through the predisposed leg extensions (22) and extended leg pieces (23). The second dual open-end needle (26) is inserted into and within a puncture stopper (37). The second dual open-end needle (26) (12) can have a length of from about half an inch to about three quarters of an inch (1.27 cm to 1.91 cm). The gauge of the needle (26) may vary while the gauge number indicates the diameter of its lumen/bore, meaning the circular hollow space inside both the first dual open-end needle (12) and the second dual open-end needle (26). Rubber sleeve cap (24) is configured to attached to the extended leg pieces (23) and secure the second dual open-end needle (26) to the splitter bridge assembly (32). The rubber sleeve (27) covers the portion of the second dual open-end needle that protrudes downward from rubber sleeve cap (24).

In some aspects, the rubber sleeve (27) described above, is designed to maintain the sterilization of the needle(s) (26). When the puncture stopper (37) of the evacuated vial(s) (39) is pushed onto or inserted onto the one end of the second dual open-end needle (26), the rubber sleeve (27) collapses. The rubber sleeve (27) acts to cover the second dual open-end needle (26) tip and to prevent leakage and contamination before and after the second dual open-end needle (26) is inserted into puncture stopper (37) mounted in evacuated tube (39).

In yet other aspects of the device, the connector or attaching means (25) for the evacuated vial holder (34) is mounted to the rubber sleeve cap (24). The connector means (25) can be located at the right of each rubber sleeve cap (24). The connector means (25) can be a snap, wherein there is a cut-out groove (31) which is positioned and located at the rim (29) of the evacuated vial holder (34), for easy one-way glide assembly. The snap is rotated to the left to accomplish final locking, secure placement and extra tight sealing between the splitter bridge system (32) and evacuated vial holder (34).

In some aspects of the device, the evacuated vial holder (34), is configured as a three leaf clover, and consist of an attaching means, such as is capable of accepting a snap (25), a puncture stopper (37) and up to a maximum of three (3) evacuated vials (39), and wherein prior to utilizing the holder as a blood collecting system, it must be properly configured with attaching means (25), such as a snap, together with the Y-configured splitter bridge (21) and evacuated vial holder (34) prior to being utilized as a blood collection system.

In other aspects of the device, when the splitter bridge system (32) is aligned with the exterior base cut out (28) at the rim (29) of the evacuated vial holder (34) the splitter bridge assembly (32) is pushed passing through the exterior base cut-out (28) into the interior locking station (30) between the vertical and horizontal walls of the evacuated vial holder neck (33) and are snapped together. The clinician will know that the two are now tightly secure and in place by the “snap” sound that indicates that the two easy glide slits (31) are in proper alignment. The clinician can now rotate the splitter (32) as one unit to the left. The splitter bridge assembly (32) now rests on the neck (33) of the evacuated vial holder (34).

In some aspects of the device, FIG. 4 shows a clover shaped evacuated vial holder (34) having a neck (33) that extends vertically while having a hollow middle core for accepting the rubber sleeves (27) during assembly. Necks (33) are mounted to the top and centered on each cone shaped compartment of the holder (34). The second dual-open-end needle (26) and rubber sleeves (27) are the only portion of the splitter bridge assembly (32) that passes through and into the holder (34) during assembly. The clinician can now proceed with the process of blood collection.

In yet other aspects of the device, the evacuated vial holder (34) is a clear vacutainer one user needle holder. The evacuated vial holder (34) can be made of any malleable material such as polypropylene and the like and can be shaped like a three leaf clover in order to accommodate the Y-configured splitter bridge and assembly (21) and (26) and the evacuated vial holder (34) may contain from one to a maximum of three vials (39).

In yet other aspects of the device, FIG. 4 shows spacers (35) that can be made of a malleable material that extends inward at creases of the evacuated vial holder (34). The spacers (35) aid in keeping the evacuated vials (39) in place. Each compartment of the holder (34) is designed to help avoid clashing of the vials (38) causing them to break or leak resulting in cross contamination and possible injury to the clinician and patient.

With the holder (34) and spacers (35) holding the evacuated vials (39) securely into place, it provides yet another safety barrier between the patient and the clinician. The evacuated vial holder (34) is designed to hold up to three 13 mm and 16 mm diameter evacuated vials (250/sp, 1000/ca), having a length of about 13 inches (33 cm) to about 17.7 inches (45 cm), a width of about 9.0 inches (23 cm) to about 13.8 inches (43 cm), a height of about 6.8 inches (17 cm) to about 12.2 inches (30 cm) and a weight of about 0.143 ounce (oz) (4 gms) to about 8.29 pounds (235 gms).

In some aspects of the device, prior to utilizing the evacuated vial holder (34) as a blood collecting system, it must be properly configured and aligned with the attaching means (25), such as the snap discussed above, in order to put together the Y-configured splitter bridge (32) and the evacuated vial holder (34) to utilize as a blood collection system.

In one aspect of the device, FIG. 4, shows an evacuated vial set-up (39) that is commonly used by healthcare professionals to draw a sample of body fluids from a patient in a hospital, doctor's office, emergency and non-emergency setting for diagnostic testing. Evacuated vial set-up (39) consists of an evacuated top (36) which is a malleable material that come in many colors, depending on what test is being performed. A puncture stopper (37) is a thick, re-sealable, penetrable rubber which is mounted on top of the evacuated top (36) and inside the vial (38). The evacuated vials (38) may contain anticoagulants and/or other chemical additives. The evacuated vial set-up (39) is used under vacuum so that blood feeds into the vial (38) as soon as the puncture stopper (37) is penetrated.

I claim:

1. A device for simultaneously collecting multiple blood samples comprising: a butterfly needle system; a splitter bridge assembly; an evacuated vial holder and evacuated vials.

2. The device according to claim 1, wherein the butterfly needle system comprises a dual open-end needle; a base for the needle, a butterfly or flexible wings, memory free tubing, and a female luer lock adaptor.

3. The device according to claim 1, wherein the splitter bridge assembly comprises a male luer lock connector system having a predisposed extension tip, an open core capillary, a male luer lock base, a Y-configured splitter bridge, predisposed leg extensions, extended pieces, rubber sleeve caps, a connector means, a dual open end needle and a rubber sleeve.

4. The device according to claim 3, wherein the second dual open-end needle is covered by a rubber sleeve prior to the evacuated vial being pushed onto the needle.

5. The device according to any one of claim 1, wherein the memory free tubing is attached to the butterfly needle system on one end to a female luer lock connector that is connected to a male luer lock connector unit that is mounted on top of the Y-configured splitter bridge.

6. The device according to claim 1, wherein the Y-configured splitter bridge is further configured with an evacuated vial holder that connects or attaches to the splitter bridge assembly.

7. The device according to claim 6, wherein the connecting or attaching means for the evacuated vial holder to the splitter bridge assembly is a snap.

8. The device according to claim 1, wherein the Y-configured splitter bridge further comprises pre-disposed leg extensions and extended leg pieces.

9. The device of claim 3, wherein the rubber sleeve cap is configured to accept a second dual open-ended needle, wherein the second dual open-ended needle is inserted into the middle of the rubber sleeve cap and the opposite end of second dual open-ended needle is inserted into puncture stopper.

10. The device according to claim 1, wherein the rubber sleeve cap is further configured with a connecting means for attaching the Y-configured splitter bridge with the evacuated vial holder.

11. The device according to claim 10, wherein the rubber sleeve cap is further configured to accept a rubber sleeve, which can cover a second dual open-ended needle mounted to the middle of rubber sleeve cap and the Opposite of the second dual open-ended needle is inserted into and through puncture stopper for the purpose of delivering blood into from one to three evacuated vials from a single insertion site.

12. The device according to claim 1, wherein evacuated vial holder is configured as a three leaf clover.

13. A method for collecting multiple blood samples from a patient using a single needle insertion site comprising:
a) inserting one end of a dual open-end needle of a butterfly needle system into the patient;
b) allowing the blood to flow through memory free tubing to a Y-configured splitter bridge; wherein the blood is evenly divided to simultaneously fill up to three evacuated vials.
